(19) United States
(12) Patent Application Publication
(10) Pub. No.: US 2010/0249694 A1
(43) Pub. Date: Sep. 30, 2010

(54) TROCAR FOR A SINGLE PORT SURGERY

(76) Inventors: Seung Wook CHOI, Seongnam-si (KR); Jong Seok Won, Yongin-si (KR); Bae Sang Jang, Hwasung-si (KR)

Correspondence Address: BIRCH STEWART KOLASCH & BIRCH, PO BOX 747, FALLS CHURCH, VA, 22040-0747, US

(21) Appl. No.: 12/712,008

(22) Filed: Feb. 24, 2010

(30) Foreign Application Priority Data
Mar. 31, 2009 (KR) 10-2009-0027850
Jun. 23, 2009 (KR) 10-2009-0055988

(51) Int. Cl. A61B 17/34; A61M 37/00

(52) U.S. Cl. 604/26; 604/164.01; 604/167.03

(57) ABSTRACT

Provided is a trocar for a single port surgery installed on a skin puncture site of a patient to provide a pathway for insertion of a plurality of surgical instruments. The trocar includes an upper part; a plurality of inserting holes configured to be positioned on the upper part, the plurality of the surgical instruments being inserted thereto; and a lower part configured to be formed in a tubular shape to connect to the upper part, the lower part being formed in an elastic material so that the lower part is bent when being inserted into the skin puncture site and is straightened after being introduced into the skin puncture site, wherein the trocar provides an open interior space in which the inserting holes are connected with one another so that changing locations of the plurality of the surgical instruments is possible after the surgical instruments are inserted into the plurality of the inserting holes.

### CROSS-REFERENCE TO RELATED APPLICATIONS

This application claims the benefit of Korean Patent Application No. 10-2009-0027850 filed with the Korean Intellectual Property Office on Mar. 31, 2009, and Korean Patent Application No. 10-2009-0055988 filed with the Korean Intellectual Property Office on Jun. 23, 2009, the disclosures of which are incorporated herein by reference in their entirety.

### BACKGROUND OF THE INVENTION

The present invention relates to a medical apparatus, and more particularly to a trocar for a single port surgery.

Surgery is a medical term that refers to a procedure that involves cutting, excising or maneuvering a patient's skin, mucous membrane, tissue or the like by using a medical device in order to treat a pathological condition such as disease. Among various surgical methods, laparotomy is a surgical procedure that involves an incision through an abdominal wall or facial skin to gain access into organs therein so as to treat, manipulate or remove the organs.

Particularly, when surgery involves laparotomy, a patient's skin is first cut open and lifted so that a certain space can be created between the patient's skin and tissue, in which surgical operation can be performed. Thus, since laparotomy requires large incisions, resulting in more pain and longer recovery time, an increasing attention has been paid to the importance of a laparoscopic surgery. Generally, in the laparoscopic surgery, an operation of an abdomen is performed through a small incision while a surgeon is able to view the operative field through the incision. The laparoscopic surgery is commonly used in various surgical sub-specialties including a gall bladder removal surgery, an appendectomy, a gastrectomy, a colectomy as well as gynecologic surgery and urology. Also, a single port surgery in which a surgeon operates almost exclusively through a single entry point is advantageous in that scarring is greatly reduced compared to the conventional laparoscopic surgery. However, the single port surgery can be a tricky maneuver to perform.

FIG. 1 illustrates a perspective view of a conventional trocar for a single port surgery. A surgical trocar is a medical equipment that is used to allow access to an abdomen during a laparoscopic surgery. By using the medical trocar, a medical tool such as a laparoscope or an endoscope is inserted into the abdomen. Referring to FIG. 1, an inserting hole 110 and a cannula 120 are illustrated. A plurality of the inserting holes 110 are formed by puncturing a protrusion positioned on an upper part of the trocar to receive a surgical instrument. In addition, the cannula 120 has a lotus root-like shape such that the inserting holes 110 are respectively connected to different internal pipes. Therefore, each surgical instrument is inserted to one of the different inserting holes 110 and then is guided through a predefined pathway.

According to the conventional art, the protrusion on which the inserting hone 110 is formed is engaged with a cover having a larger diameter. Therefore, the protrusion can often come into contact with other adjacent protrusion. In addition, in the conventional trocar, in order to change the location of a particular surgical instrument, which has already been introduced, every surgical instrument placed within the trocar has first to be removed from the respective inserting hole 110 so that the particular surgical instrument can be inserted to the desired inserting hole 110.

The above information disclosed in this Background section is retained or acquired by the inventor in an effort to realize the object of the invention, and therefore it may contain information that does not form the prior art that is already known to the public.

### BRIEF SUMMARY OF THE INVENTION

The present invention has been made in view of the above problems, and provides a trocar for a single port surgery which has increased convenience in changing the location of a surgical instrument after the surgical instrument is introduced thereto.

The present invention further provides a trocar for a single port surgery which has increased convenience in engaging and/or disengaging the surgical instrument from the trocar.

According to the present invention, a trocar for a single port surgery includes: an upper part on which a plurality of inserting holes are positioned while the plurality of the surgical instruments are inserted to the inserting holes; and a lower part configured to be formed in a tubular shape having an empty space therein in which the inserting holes are connected with one another, the lower part having a first end to be inserted into a skin puncture site.

Here, the trocar may further include a valve configured to engage with the inserting hole to prevent an air flow. The valve may be formed to have a certain shape such as, for example, S, +, −Y or a rib of a fan. The valve may be formed integrally with the inserting hole.

The upper part may include a cover that covers the inserting hole, wherein a hole formed in the cover. The cover may be formed in an elastic member, which can be one of rubber, silicone and elastic fiber.

The upper part may include an upper part ring for maintaining a shape of the upper part and the lower part may include a lower part ring, which has elasticity such that the lower part ring is bent when being inserted into the skin puncture site and is straightened after being introduced into the skin puncture site.

In one example embodiment, the trocar may further include an injection tube configured to inject a gas from an external environment into inside of the skin puncture site, an exhaust tube configured to exhaust a gas from the inside of the skin puncture site to the external environment, and an opening/closing valve configured to engage with at least one of the injection tube and the exhaust tube to control a flow of the gas.

In addition, in one example embodiment, the trocar may further include a sensor that is configured to engage with a first end of the lower part and is configured to detect the gas in the inside of the skin puncture site to produce a valve opening/closing signal and a valve opening/closing device configured to determine whether to operate the opening/closing valve based on the valve opening/closing signal received from the sensor.

The upper part and the lower part are separate tubes that are separable from each other and at least one of the injection tube and the exhaust tube extends to a first end of the lower part.

At least one of the upper part and the lower part is formed in a flexible material such as vinyl or silicone.

The upper part and the lower part can be formed integrally or separably from each other. In one example embodiment, the trocar may further include an engaging ring configured to engage a first end of the upper part with a second end of the lower part. The engaging ring is a separate ring that is separable from the upper part and the lower part. The engaging ring may include an internal ring and an external ring configured to engage with the internal ring along an outer periphery of the internal ring.

At least one of the internal ring and the external ring may have an increased surface roughness, and the internal ring and the external ring may be engaged with each other in a concave-convex engagement. The engaging ring may be formed integrally with the upper part or the lower part.

Also, the engaging ring may include an upper part ring configured to engage with the upper part and a lower part ring configured to engage with the lower part, the lower part ring being drawn to the upper part ring by an attractive force caused by magnetism.

In addition, in one example embodiment, the trocar may further include a fixing ring configured to enclose a surrounding of the lower part to fix a state of the lower part being inserted through the skin puncture site.

The inserting hole may be configured to have a certain level of a hardness such that the inserting hole is not distorted or deformed by an external force during a use of the trocar. To this end, the inserting hole may be engaged with a distortion preventing member having the certain level of the hardness. Also, the trocar may further include a valve configured to engage with the inserting hole to block an air flow, wherein a portion of the valve to be engaged with the inserting hole is configured to have a certain level of a hardness such that the inserting hole is not distorted or deformed by an external force during a use of the trocar.

The upper part and the lower part may be integrally formed with each other, and a fastening member may be formed inside the trocar to fasten a tube that provides a passageway for an air between an external environment and inside of the skin puncture site.

### BRIEF DESCRIPTION OF THE DRAWINGS

FIG. 1 illustrates a perspective view of a conventional trocar for a single port surgery.

FIG. 2 illustrates a perspective view of a trocar for a single port surgery according to one example embodiment of the present invention.

FIG. 3 illustrates a front view of a trocar for a single port surgery according to one example embodiment of the present invention.

FIG. 4 illustrates a front view of a trocar for a single port surgery according to one example embodiment of the present invention.

FIG. 5 illustrates a cross-sectional view of an engaging ring of a trocar for a single port surgery according to one example embodiment of the present invention.

FIG. 6 illustrates a front view of a trocar for a single port surgery according to one example embodiment of the present invention.

FIG. 7 illustrates a front view of a trocar for a single port surgery according to one example embodiment of the present invention.

FIG. 8 illustrates a front view of a trocar for a single port surgery according to one example embodiment of the present invention.

FIGS. 9 and 10 illustrate a state where a medical instrument is inserted to a trocar for a single port surgery according to one example embodiment of the present invention.

FIGS. 11A through 11H illustrate a structure of an inserting hole according to one example embodiment of the present invention.

FIG. 12 illustrates a cross-sectional view of a fastening unit according to one example embodiment of the present invention.

FIGS. 13A through 13C illustrate magnifying perspective views of a fastening unit according to various example embodiments of the present invention.

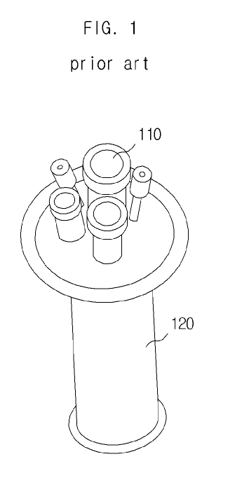

### DESCRIPTION OF EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein. Accordingly, example embodiments are to cover all modifications, equivalents, and alternatives falling within the scope of the invention.

It will be understood that, although the terms first, second, etc. may be used herein to describe various elements, these elements should not be limited by these terms. These terms are only used to distinguish one element from another.

It will be understood that, when a feature or element is referred to as being “connected” or “coupled” to another feature or element, it can be directly connected or coupled to the other element or intervening elements may be present. In contrast, when a feature or element is referred to as being “directly connected” or “directly coupled” to another element, there are no intervening elements present.

The terminology used herein is for the purpose of describing particular embodiments only and is not intended to be limiting of example embodiments of the invention. It will be understood that the terms “comprises,” or “includes,” when used herein, specify the presence of stated features, integers, steps, operations, elements, and/or components, but do not preclude the presence or addition of one or more other features, integers, steps, operations, elements, components, and/or groups thereof.

Like numbers are used throughout the drawings to refer to the same or like parts and a repetitive explanation will be omitted. Detailed descriptions of well-known functions and structures incorporated herein may be omitted to avoid obscuring the subject matter of the present invention.

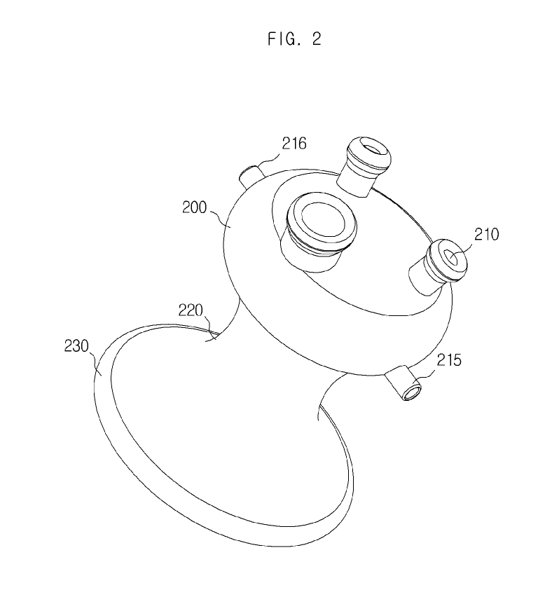

FIG. 2 illustrates a perspective view of a trocar for a single port surgery according to one example embodiment of the present invention. Referring to FIG. 2, an upper part 200, an inserting hole 210, an injection tube 215, an exhaust tube 216, a lower part 220 and a lower part ring 230 are illustrated.

According to the example embodiment of FIG. 2, a plurality of surgical instruments are inserted into different inserting holes 210 to be introduced into an abdomen of a patient through the same path. In other words, in the surgical trocar according to this example embodiment, the plurality of the inserting holes 210 are connected at a single open interior space at a bottom portion of the upper part 200 on which the plurality of the inserting holes 210 are located. Therefore, it is easy and convenient to change the location of the surgical instruments after the insertion of the surgical instruments into the inserting holes 210.

The number of the inserting holes 210 formed in the upper part 200 can be determined depending on the type of surgery to be performed. The inserting hole 210 can be protruded from the upper part 200 by a predetermined length. In this manner, the insertion of the surgical instruments can be facilitated as well as the installation of a valve, which will be described below. Each of the inserting holes 210 is spaced apart from one another by a predetermined distance so that the inserting holes 210 are not in contact with one another.

The upper part 200 can be formed in a flexible material such as vinyl or silicone. When the upper part 200 is formed in the flexible material, the surgical instruments can be inserted from various angles. Also, the surgical instruments can move freely after placing the surgical instruments into the trocar.

At the bottom portion of the upper part 200, an open space is formed wherein a boundary of the open space can be formed in various shapes such as circle, oval, etc. Also, the upper part 200 may further include an upper part ring (not shown) for maintaining the shape of the upper part 200. That is, when the upper part 200 is formed in an amorphous material such as vinyl, the upper part ring can be used in order to maintain the shape of the upper part 200 so that the pathway of the surgical instrument can be clearly defined. For example, the upper part ring can be configured to enclose the outer boundary of the upper part 200 so that sufficient internal space of the upper part 200 is secured.

The injection tube 215 is used to supply gas (e.g., carbon dioxide) that is needed to perform an operation to the abdomen of a patient. The exhaust tube 216 is used to vent gas in the abdomen to an external space, for example, a vacuum suction pipe or a ventilator of a surgery room. Thus, the injection tube 215 injects gas from the external space into the body through a skin puncture site that is prepared for a surgery and the exhaust tube 216 emits the gas from the inside of the body to the external space through the skin puncture site. The injection tube 215 and the exhaust tube 216 can be positioned in different locations, for example, opposite ends of the upper part 200, so that the respective gas flows in the injection tube 215 and the exhaust pipe 216 may not be affected by each other.

An opening/closing valve can be installed at the injection tube 215 and/or the exhaustion tube 216 to control the gas flow therein. The gas flow is allowed when the opening/closing valve is opened and the gas flow is prohibited when the valve is closed. In this manner, the opening/closing valve may control the flow of the gas.

The lower part 220 can be formed in a pipe having an open empty space so that the inserting holes 210 can be connected thereto. A first end of the lower part 220 is inserted into the skin puncture site. A central part of the trocar can be formed as a pipe of which both sides are concave. That is, when the lower part 220 is inserted through the skin puncture site, the skin puncture site extends around the concave sides of the central part of the trocar formed in the manner described above so that the first end of the lower part 220 and the upper part 200 can support the surgical trocar according to one example embodiment of the invention. The lower part 220 can also be formed in a flexible material such as vinyl or silicone.

The upper part 200 and the lower part 220 can be formed integrally with each other or provided in separate components. When the upper part 200 and the lower part 220 are integrally formed, the inserting hole 210 may be formed in a first end of the body and a second end of the body may be inserted into the skin puncture site. That is, the bottom portion of the upper part 200 having the plurality of the inserting holes 210 to which the surgical instrument are inserted is configured to be formed in a pipe having an open empty space therein as described above so that modifying the location of the surgical instrument is convenient after the surgical instrument is placed in the trocar.

Also, when the upper part and the lower part 220 are provided in separate components, the engaging structure between the upper part 200 and the lower part 220 can be embodied in various ways. For example, the upper part 200 and the lower part 220 can be engaged with each other by using a ring having a predetermined shape or by using a magnet prepared in each component of the upper part 200 and the lower part 220.

The lower part ring 230 can be further included. The lower part ring 230 can be located in one end of the lower part 220, wherein the lower part ring 230 has elasticity such that the lower part ring 230 is bent when being inserted into the skin puncture site and is straightened after being introduced into the skin puncture site. The lower part ring 230 can be formed in a material having elasticity such as rubber, silicone or elastic fiber. Therefore, a doctor can bend the lower part ring 230 to insert one end of the lower part 220 through the skin puncture site on the patient.

When the upper part 200 and the lower part 220 are integrally formed with each other, the doctor can insert the surgical instrument into the inserting hole 210 immediately after inserting the lower part 220 into the patient's body to perform the operation. In contrast, when the upper part 200 and the lower part 210 are formed in separate members, an additional step is needed to engage the upper part 200 with the lower part 220 that is inserted into the skin puncture site.

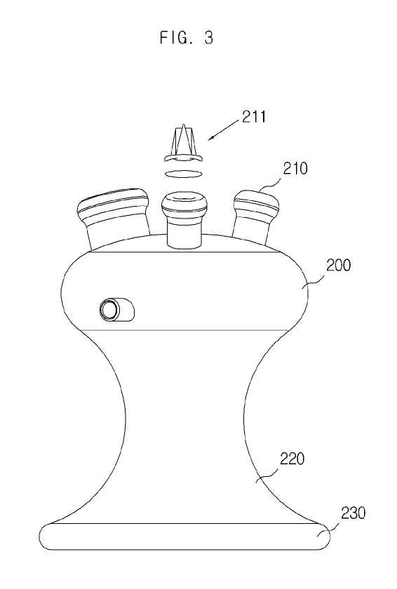

FIG. 3 illustrates a front view of a trocar for a single port surgery according to one example embodiment of the present invention. Referring to FIG. 3, the upper part 200, the inserting hole 210, a valve 211, a sealing member 212, a cover 213, a cover hole 214, the exhaust tube 216, the lower part 220 and the lower part ring 230 are illustrated. The description below will be mainly focused on the difference between the embodiments of FIGS. 2 and 3.

In the embodiment of FIG. 3, the valve 211 and the cover 213 are used in order to solve the problem that an internal gas can be discharged to the outside through the surgical instrument that is inserted into the inserting hole 210. Namely, when the surgical instrument is inserted, the valve 211 firstly prevents the gas leakage and then the cover 213 also blocks the gas flow.

The inserting hole 210 can be engaged with the valve 211, which prevents an air flow. The valve 211 includes the sealing member 212 for preventing the air flow and one end of the sealing member 212 is closed in a certain manner. One end of the sealing member 212 can be formed to have a certain shape such as, for example, S, +, −Y or a rib of a fan. To this end, the sealing member can be distorted so that one end of the sealing member can be formed to have a particular shape. Alternatively, an appendage having the particular shape can be placed in one end of the sealing member 212. Here, the forming of the rib of a fan frame can be accomplished by overlapping S frames or Yin-Yang symbols. Since the valve that prevents an air leakage is well known to those skilled in the art, further description will thus be omitted.

The valve 211 and the inserting hole 210 can be formed in one body or in separate members. When the valve 211 and the inserting hole 210 are formed in one body, the valve 211 is not separable from the inserting hole 210 so that the valve 211 does not need to be managed separately. Also, when the valve 211 is separably engaged with the inserting hole 210, the valve 211 only needs to be inserted to the inserting hole 210 to prevent the air flow.

The cover 213 is positioned at a top portion of the valve 211 and is used to cover the inserting hole 210. That is, the effect of decreased air flow by the valve 211 can be limited so that the cover 213 can also be used to block the air flow. The cover 213 is punctured to form the cover hole 214 to allow the insertion of the surgical instrument thereto.

The cover 213 can be formed from an elastic material. Thus, when the surgical instrument is inserted or moves, the cover 213 can be tightened to block the air flow. Herein, the elastic material may include rubber, silicone and elastic fiber.

For example, when the surgical instrument is not inserted into the inserting hole 210, the valve 211 is closed so that the air is not leaked out. When the surgical instrument is inserted to the inserting hole 210, the valve 211 is opened as well as the sealing member 212 so that there exists a possibility that the air leaks out. However, by using the cover 213 having the cover hole 214 of which diameter is smaller than the diameter of the surgical instrument, the air leakage can be prevented.

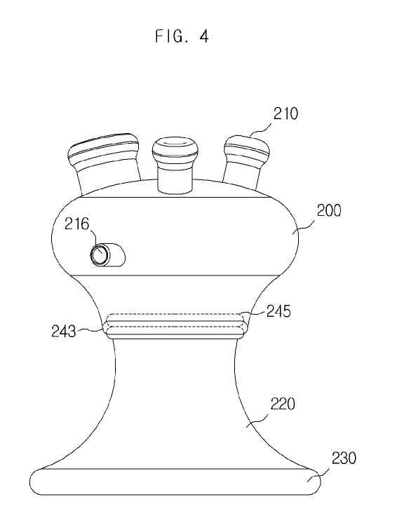

FIG. 4 illustrates a front view of a trocar for a single port surgery according to one example embodiment of the present invention. Referring to FIG. 4, the upper part 200, the inserting hole 210, the exhaust tube 216, the lower part 220, the lower part ring 230, an external ring 243 and an internal ring 245 are illustrated. Discussion in the below will be mainly focused on the difference between the embodiment of FIG. 2 and other embodiments described above.

In the example embodiment of FIG. 4, the upper part 200 and the lower part 220 are engaged in a separable manner. Also, the upper part 200 and the lower part 220 are engaged with each other by using the engaging rings 243 and 245. Namely, the engaging rings 243 and 245 are used to engage one end of the upper part 200 with one end of the lower part 220.

The engaging rings 243 and 245 can be configured to be integrally formed with the upper part 200 and/or the lower part 220. Alternatively, the engaging rings 243 and 245 can be configured to be separable from the upper part 200. Herein, in the case of integrated housing, the engaging rings 243 and 245 are not intended to be separated from the upper part 200 and/or the lower part 220 during a normal use of the trocar and the doctor cannot easily separate the engaging rings 243 and 245 from the upper part 200 and/or the lower part 220. The engaging rings 243 and 245 do not need to be formed in the same material as the upper part 200 and/or the lower part 220.

The engaging ring can be formed in an elastic member having flexibility. When the engaging ring includes the external ring 243 and the internal ring 245, the external ring 243 is engaged with the internal ring 245 along an outer periphery of the internal ring 245. Referring to FIG. 4, a groove is formed along the outer periphery of the internal ring 245 and the external ring 243 is engaged with the groove of the internal ring 245. Such engagement is referred to as a concave-convex engagement. Alternatively, a groove can be formed along an outer periphery of the external ring 243 and the internal ring 245 can engage with the groove of the external ring 243.

Herein, the external ring 243 is coupled to one end of the upper part 200 and the internal ring 245 is coupled to one end of the lower part 220. Therefore, the upper part 200 is coupled to the lower part 220 by coupling of the external ring 243 and the internal ring 245.

In addition, the external ring 243 and/or the internal ring 245 may have a higher surface roughness. When the external ring 243 and/or the internal ring 245 have a higher surface roughness, it is advantageous in that the external ring 243 and the internal ring 245 are not easily separable from each other once the external ring 243 is coupled to the internal ring 245.

In addition, according to an alternative embodiment, the engaging ring can be implemented in a pair of magnet or any material that has a magnetic force to ease the engagement between the engaging rings. For example, an upper engaging ring (not shown) can be engaged with the upper part 200 and a lower engaging ring (not shown) can be engaged with the lower part 220, wherein the upper and the lower engaging rings are engaged with each other by the magnetic force, thereby engaging the upper part 200 and the lower part 220. Here, the upper engaging ring and the lower engaging ring can be magnets having different magnetic poles. Alternatively, one of the upper engaging ring and the lower engaging ring can be a magnet while the other is a material that moves toward a magnet, for example, a metal including iron, nickel or alloy thereof.

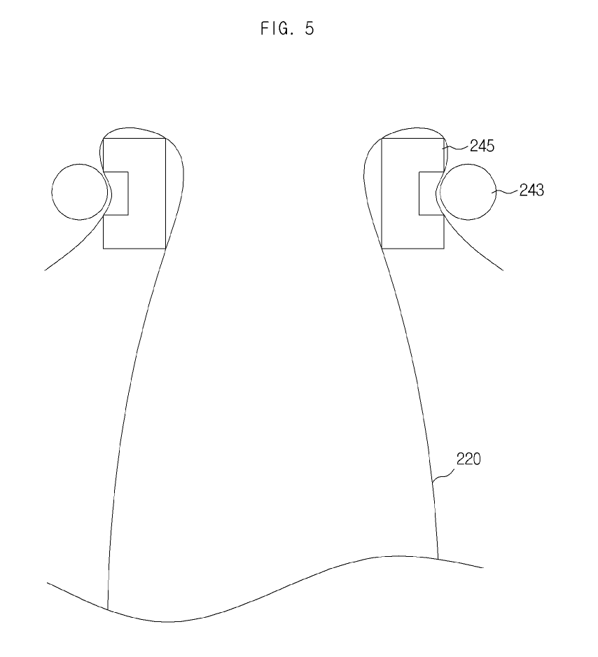

FIG. 5 illustrates a cross-sectional view of an engaging ring of a trocar for a single port surgery according to one example embodiment of the present invention. Referring to FIG. 5, the lower part 220, the external ring 243 and the internal ring 245 are illustrated.

In the embodiment of FIG. 5, when the lower part 220 is formed in an amorphous material such as vinyl, the external ring 243 and the internal ring 245 are used to help one end of the lower part 220 to form a specific shape as well as configure the lower part 220 to be engageable with the upper part 200.

According to this embodiment, the internal ring 245 receives the lower part 220 and the external ring 243 is coupled to the internal ring 245 with the lower part 220 being interposed therebetween. Namely, the lower part 220 passes through the inside of the internal ring 245 to surround the exterior of the internal ring 245 and is positioned between the external ring 243 and the internal ring 245. In this way, the positioning of the lower part 220 can be fixed.

Also, a groove can be formed along an outer periphery of the internal ring 245 to facilitate the coupling of the external ring 243 and the internal ring 245. Although not shown in the drawings, it should be noted that a groove can be formed along an inner periphery of the external ring 243 to facilitate the coupling the external ring 243 and the internal ring 245.

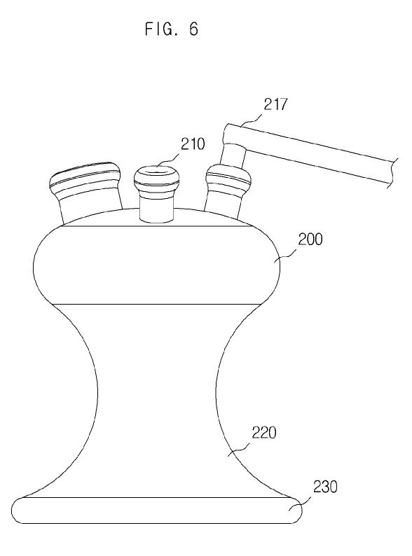

FIG. 6 illustrates a front view of a trocar for a single port surgery according to one example embodiment of the present invention. Referring to FIG. 6, the upper part 200, the inserting hole 210, a separable type injection tube 217, the lower part 220 and the lower part ring 230 are illustrated.

In the embodiment of FIG. 6, the injection tube 217 for injecting gas is prepared as a separate tube that can be separated from the trocar. The injection tube 217 is not formed on the upper part 200 in one body as described above but is instead provided in a separate tube, for example, a rubber tube, to enable the control of the air flow. The separable type injection tube 217 is inserted into the inserting hole to inject a certain gas. When the injection of gas is no longer wanted, the injection tube 217 can be removed from the inserting hole 210. Thus, the separable-type injection tube 217 can be used flexibly depending on the needs. It should be noted that the exhaust tube can also be configured as a separable-type tube.

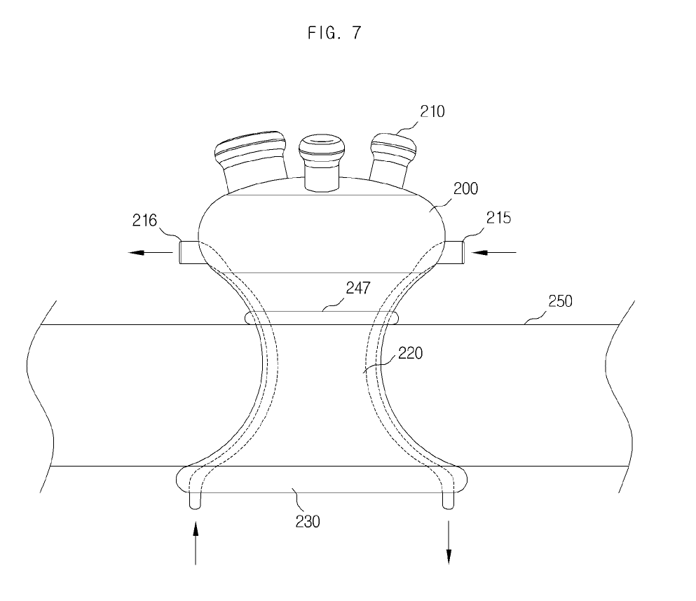

FIG. 7 illustrates a front view of a trocar for a single port surgery according to one example embodiment of the present invention. Referring to FIG. 7, the upper unit 200, the inserting hole 210, the injection tube 215, the exhaust tube 216, the lower part 220, the lower part ring 230, a fixing ring 247 and a skin 250 of a patient are illustrated. The discussion below will be mainly focused on the difference between the embodiment of FIG. 7 and other embodiments described above.

In the embodiment of FIG. 7, one end of the injection tube 215 and/or one end of a first end of the exhaust tube 216 is protruded from the upper part 200 and the other end of the injection tube 215 and/or the other end of the exhaust tube 216 extends near the lower part ring 230, thereby efficiently controlling the gas flow in the abdomen of the patient. That is, if the injection tube 215 and/or the exhaust tube 216 is not configured to extend as illustrated in FIG. 7 but is located only on the upper part 200, the gas in the abdomen may not be efficiently emitted. Also, the gas provided through the injection tube 215 may flow directly to the exhaust tube 216. Therefore, in this example embodiment, in order to efficiently remove the gas in the abdomen, the injection tube 215 and/or the exhaust tube 216 extends through the abdomen.

In addition, the fixing ring 247 encloses the surrounding of the lower part 220 to fix the state of the lower part 220 being inserted through the skin puncture site. The fixing ring 247 pushes on to a top surface of the skin 250 and the lower part ring 230 supports a bottom surface of the skin 250 so that the lower part 220 can be tightly fitted to the puncture site of the skin 250.

In this case, in order to provide a rigid coupling between the fixing ring 247 and the lower part 220, the fixing ring 247 may have an increased surface roughness. The surface roughness of the fixing ring 247 may be increased by forming a projection or a wrinkle on the surface of the fixing ring 247.

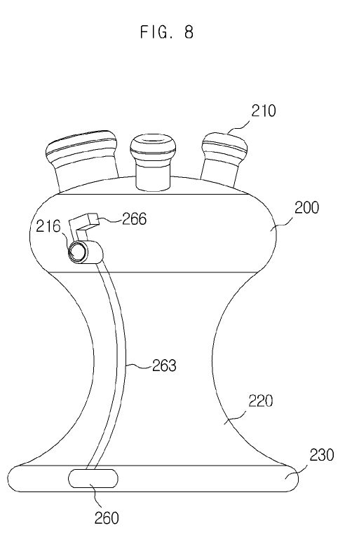

FIG. 8 illustrates a front view of a trocar for a single port surgery according to one example embodiment of the present invention. Referring to FIG. 8, the upper part 200, the inserting hole 210, the exhaust tube 216, the lower part 220, the lower part ring 230, a sensor 260, a wire line 263 and a valve opening/closing switch device 266 are illustrated.

In the example embodiment of FIG. 8, by sensing the gas inside the abdomen, the condition of the abdomen is detected and the gas inside the abdomen is promptly emitted depending on the detected condition. To this end, the sensor 260 is used to sense the existence of gas inside the body and the pressure level thereof when the trocar according to the present invention is inserted into the body. For example, when performing an electric surgery where skin burn may happen, the sensor 260 may detect if a toxic gas such as carbon monoxide is generated due to the skin burn and may produce a valve opening/closing signal upon detecting such event.

The sensor 260 can detect the gas in various methods. For example, the sensor 260 may be an electrochemical constant voltage sensor, semiconductor-type or catalytic combustion type sensor. Here, an output value of the sensor 260 may be proportional to the concentration of the gas.

Also, the sensor 260 may determine the characteristic of the valve opening/closing signal by utilizing its own mechanism. Alternatively, the sensor 260 may transfer the valve opening/closing signal to the valve opening/closing device 266 through the wire line 263 so that the valve opening/closing device 266 may determine the characteristic of the valve opening/closing signal.

The valve opening/closing device 266 may open or close the valve (the opening/closing valve) of the exhaust tube 216 such that the valve of the exhaust tube 216 may electrically and mechanically control the gas flow. Various kinds of valves can be used for the valve of the exhaust tube 216 according to example embodiments of the present invention. For example, the valve may be a stop valve in which a valve disc pushed/pulled in a direction perpendicular to a valve seat by the aid of the valve rod or a gate valve in which the valve disc moves perpendicular to the gas flow.

If the sensor 260 determines the characteristic of the valve opening/closing signal by its own mechanism, upon detecting the gas having concentration equal to or greater than a first predetermined level (a reference level), the sensor 260 may set the valve opening/closing signal as a valve on signal and transfer the valve on signal to the valve opening/closing device 266. In addition, upon detecting the gas having concentration equal to or less than a second predetermined level, the sensor 260 may set the valve opening/closing signal as a valve off signal and transfer the valve off signal to the valve opening/closing device 266.

Also, if the valve opening/closing device 266 determines the characteristic of the valve opening/closing signal, the sensor 260 transfers to the valve opening/closing device 266 the valve opening/closing signal that is the output value of the sensor 260. The valve opening/closing device 266 refers// to the output value received from the sensor 260 to determine the characteristic of the valve opening/closing signal. That is, when the received output value is equal to or greater than a first preset value, the valve opening/closing device 266 sets the characteristic of the valve opening/closing signal as the valve on signal. When the received output value is equal to or less than a second preset value, the valve opening/closing device 266 sets the characteristic of the valve opening/closing signal as the valve off signal.

By using the valve opening/closing signal, the valve opening/closing device 266 may have three operational modes. That is, the three modes include an always-closed mode, an always-open mode and an automatic mode in which the opening/closing of the valve is determined depending on the concentration of the gas as described above.

Therefore, according to example embodiments of the present invention, it is determined whether to emit the gas depending on the existence of toxic gas or the concentration thereof. Thus, the concentration of gas such as carbon dioxide injected into the abdomen of the patient during a laparoscopic surgery can be maintained at a predetermined level.

The sensor 260 is attached to the first end of the lower part 220 in an attachable/detachable manner. In this case, a connecting electrode can be formed in the sensor 260 in correspondence with an electrode that is formed in the first end of the lower part 220 in order to connect the sensor 260 and the wire line 263. Also, the valve opening/closing device 266 may also be configured to be attachable/detachable with respect to the upper part 200 or the exhaust tube 216. Generally, the surgical trocar is used as a disposable product, and therefore, adopting such an attachable/detachable structure is advantageous in that the sensor 260 and the valve opening/closing device 266 can be recycled.

The opening/closing valve of the exhaust tube 216 has the same function and structure as described in the above. Also, the opening/closing valve of the injection tube 215 can be controlled by the valve opening/closing device 266 to feed an additional gas. The sensor 260 senses the pressure level of the gas injected into the abdomen and, upon detecting when a measured pressure level is lower than a first preset reference value, the opening/closing valve of the injecting tube 215 is opened by the valve opening/closing device 266 to allow injection of the gas. When the measured pressure level is greater than a second preset reference value, the opening/closing valve of the injecting tube 215 is closed by the valve opening/closing device 266 to stop the injection of the gas.

For example, when carbon monoxide (CO) and carbon dioxide (CO2) exist inside the abdomen, partial pressure of each gas is measured by the sensor 260. If the partial pressure of the carbon monoxide is greater than a first predetermined reference value, the gas in the abdomen is emitted through the exhaust tube 216. If the partial pressure of the carbon dioxide is less than a second predetermined reference value, additional carbon dioxide is injected to the abdomen through the injection tube 215. The first and second reference values for respectively determining whether to emit or inject carbon monoxide and the carbon dioxide can be determined in advance to provide optimal conditions for surgery or determined in real time through a user interface.

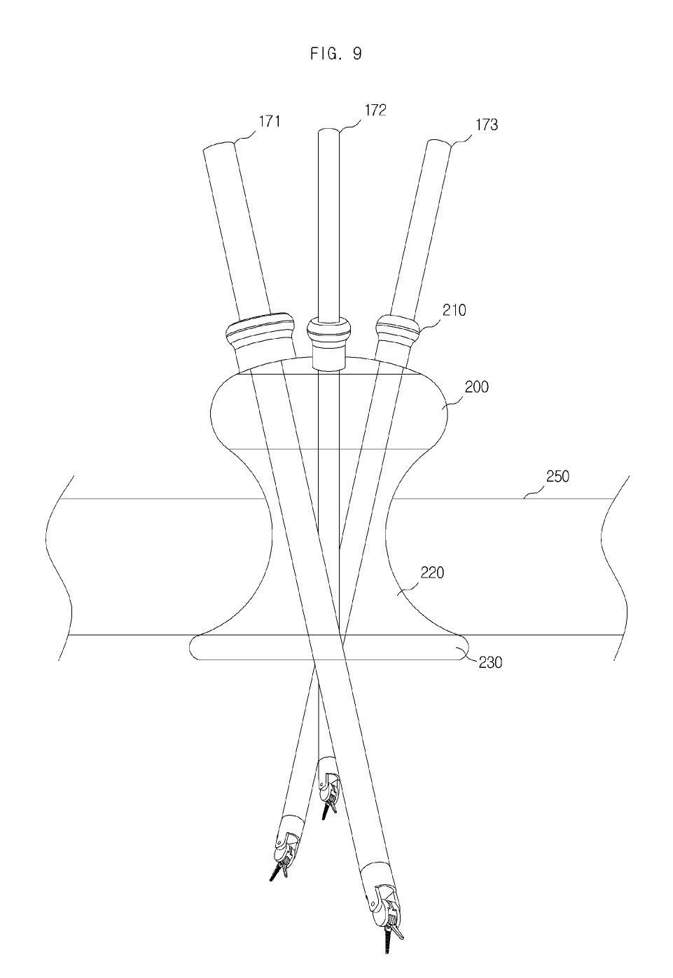

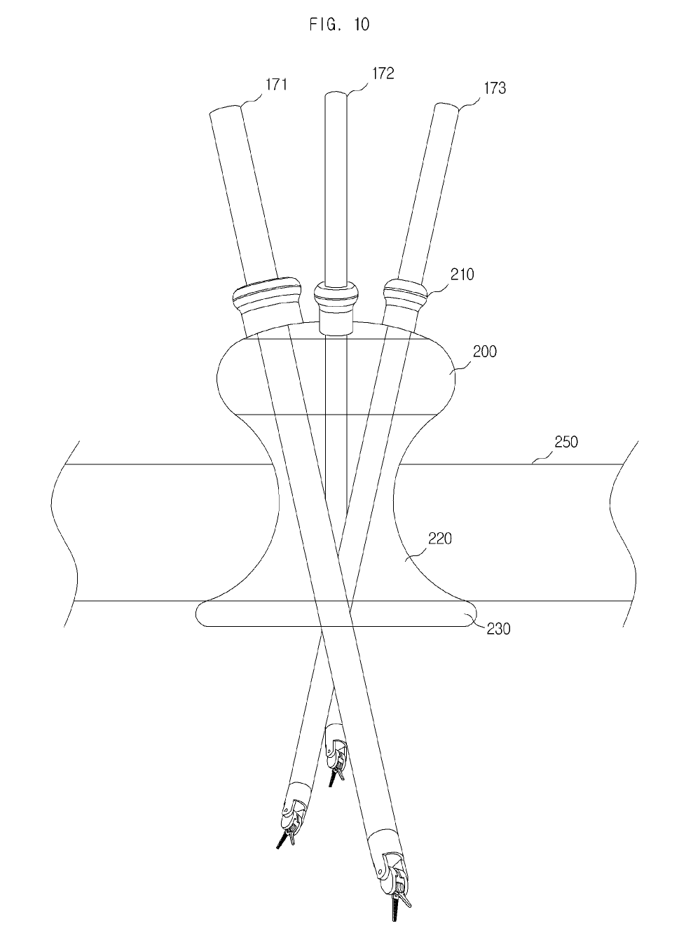

FIGS. 9 and 10 illustrate a state where a medical instrument is inserted to a trocar for a single port surgery according to one example embodiment of the present invention. Referring to FIGS. 9 and 10, three surgical instruments are inserted to the trocar according to the example embodiment of the invention. In FIG. 9, the order of placement when viewed from the front is the first instrument 271, the second instrument 272 and the third instrument 273. However, in FIG. 10, the order of placement is the first instrument 271, the third instrument 273 and the second instrument 272. Namely, since the upper part 200 to which the surgical instruments are inserted has an open empty space as described above, it is easy to change the location of the surgical instrument once the surgical instrument is placed in the trocar.

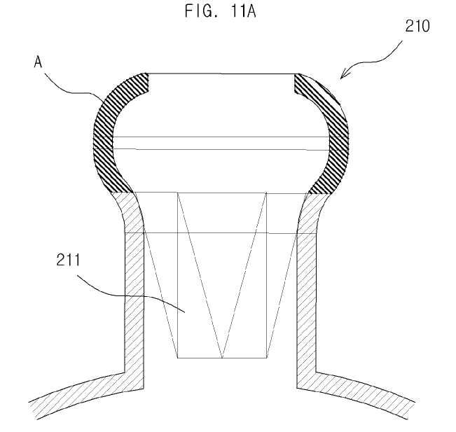

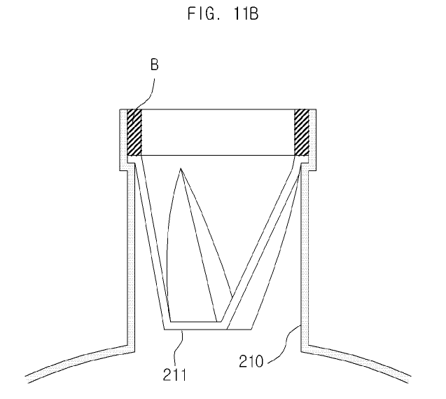

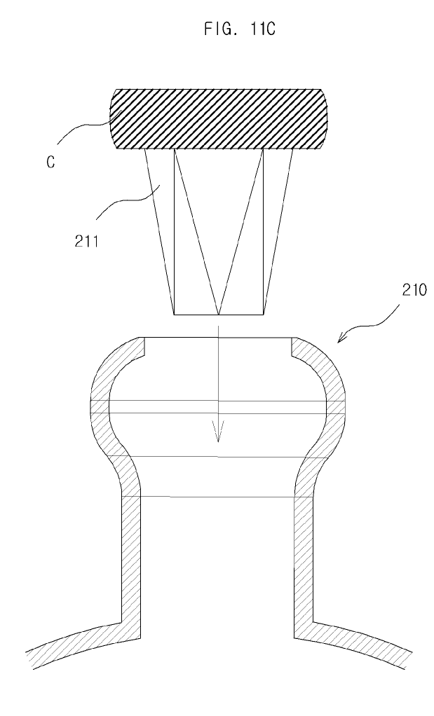

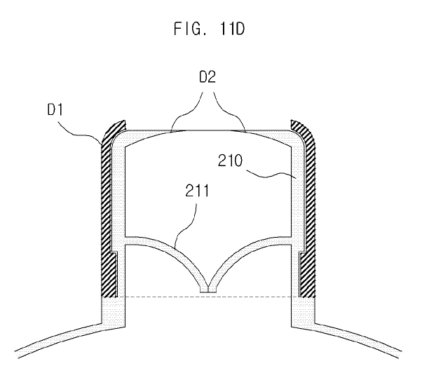

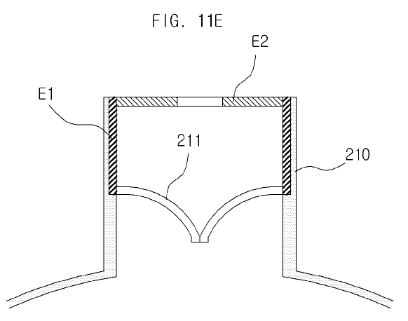

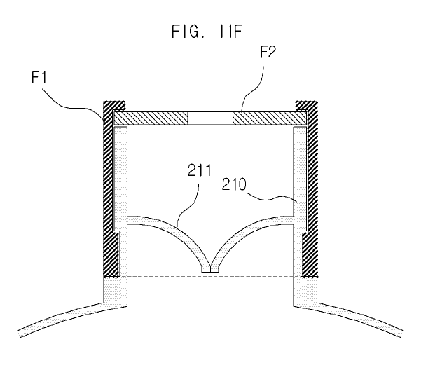

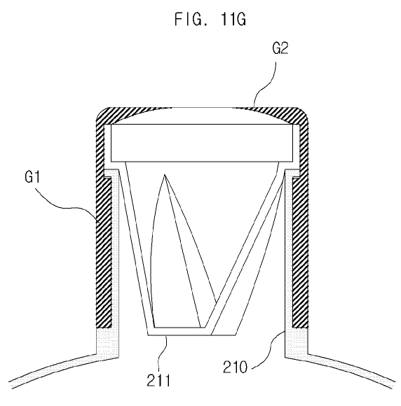

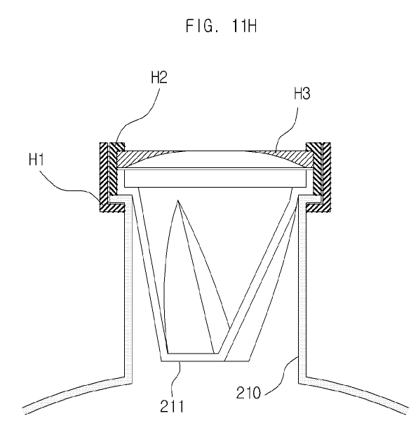

FIGS. 11A through 11H illustrate a structure of an inserting hole according to one example embodiment of the present invention. Referring to FIGS. 11A through 11H, the inserting hole 210 and the valve 211 are illustrated.

Since the trocar according to example embodiments of the present invention may be implemented in a flexible material such as vinyl or silicone, the inserting hole can be deformed by an external force during the process of installing the trocar and inserting the surgical instrument through the inserting holes. For example, if a cross section of the inserting hole is a circle, the cross section of the inserting hole can be altered to an oval or ellipse shape by the external force. This may cause unwanted air leak in or out of a patient's body through the deformed inserting hole, which may result in an erroneous operation of the valve that is installed in the trocar in order to prevent the air flow.

In order to prevent such circumstances from occurring, in the trocar according to this example embodiment, a mouth part, A in FIG. 11A, of the inserting hole 210 can be implemented in a material having a predetermined hardness. Alternatively, a distortion preventing member, B in FIG. 11B, can be positioned inside the mouth part so that the inserting hole 210 may not be deformed by the external force. It should be noted that the distortion preventing member B may not only be positioned inside the mouth part but can also be located at any other position. For example, the distortion preventing member can be inserted into the inside of the mouth part or attached to an outer surface of the mouth part.

Here, the material having a predetermined hardness can be the same material as the trocar except that the material has a hardness higher than that of the trocar. Otherwise, the material can be a copolymer material such as an acrylonitrile butadiene styrene (ABS) copolymer, which has a higher hardness than the material of the trocar, or metal such as aluminum.

For example, during the process of manufacturing the trocar by molding silicone according to example embodiments of the present invention, the mouth portion, A in FIG. 11A, of the inserting hole 210 can have a shore hardness corresponding to about 70 to about 80 such that the hardness of the mouth part is higher than any other part. Otherwise, an injection-molded material forming the distortion preventing member can be provided and engaged with the mouth part, wherein the injection-molded material is an ABS copolymer having a hardness higher than silicone and engaged with the mouth part.

Meanwhile, the valve 211 may not be installed as illustrated in FIG. 11C. Instead, the valve 211 can be manufactured as a separate member and a portion C in FIG. 11C of the valve 211 that is to be engaged with the inserting hole 210 can be configured to have an increased hardness. Thus, by fastening and attaching the valve 211 to the inserting hole 210, the inserting hole 210 may be prevented from being distorted or deformed by the external force. The material having the increased hardness can be used for a part or all of the valve member 211.

Meanwhile, when the valve 211 is installed integrally with the inserting hole as illustrated in FIG. 11D, the distortion preventing member, D1 in FIG. 11D, that is formed in a material having a higher hardness as described above can be engaged and attached to the surrounding of the inserting hole 210 so that the inserting hole 210 may not be distorted by the external force during the use of the trocar. Here, a cover, D2 in FIG. 11D, having a wing shape can be integrally formed in the mouth part of the inserting hole 210 so that the cover D2 first blocks the air flow during the insertion of the instrument and then the valve 211 also blocks the air flow.

In addition, as illustrated in FIG. 11E, the distortion preventing member, E1 in FIG. 11E, the valve 211 and the cover, E2 in FIG. 11E, can be formed as a single integral member and separately from the inserting hole 210. This single integral member can be inserted and attached to an inner side of the inserting hole 210 so that the inserting hole 210 may not be deformed by the external force during the use of the trocar. The cover and the valve have the same function as the example embodiments described in the above.

In addition, the cover may not be formed in one body with the trocar as illustrated in FIG. 11D. Instead, the cover, F2 in FIG. 11F, can be formed as a separate member to cover the inserting hole 210. The distortion preventing member, F1 in FIG. 11F, can be engaged and attached to the surroundings of the inserting hole 210 and the cover F2.

In FIGS. 11D through 11F, the inserting holes 210, the valve 211 and the cover, D2, E2 or F2, can be, for example, formed in silicone having hardness of about 30 to about 40. The distortion preventing member can be formed in silicone or ABS copolymer having hardness of about 70 to about 80 and can be bonded to the inserting hole 210.

Meanwhile, as illustrated in FIG. 11G, the valve 211 can be formed as a separate member and inserted into the inserting hole 210 wherein the distortion preventing member, G1 in FIG. 11G, can be engaged and attached to the surroundings of the inserting hole 210 and the valve 211. Also, the cover, G2 in FIG. 11G, may be formed in one body with the distortion preventing member G1 as illustrated in FIG. 11G. It should be noted that, in the embodiment of FIG. 11G, the valve may not be formed as a separate member but can be formed integrally with the trocar, particularly, the inserting hole 210.

Further, as illustrated in FIG. 11H, the valve 211, which is manufactured as a separate member, can be inserted to the inserting hole 210. The cover, H3 in FIG. 11H, which is manufactured as a separate member, can be used to cover the inserting hole 210. The distortion preventing member, H1 in FIG. 11H, can be engaged and attached to the surrounding structure of the inserting hole 210. In this case, in order to fasten the distortion preventing member H1 to the valve 211, a separate engaging member, H2 in FIG. 11H, can be interposed between the distortion preventing member and the valve 211. The engaging member H2 can be engaged with the distortion preventing member in various ways such as screw-fitting or adjusted fitting, so that the valve 211 and the cover H3 are fastened to the inserting hole 210 of the trocar.

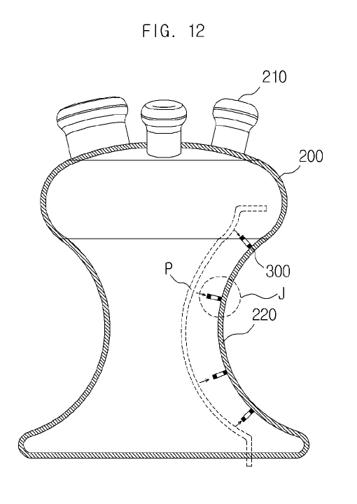

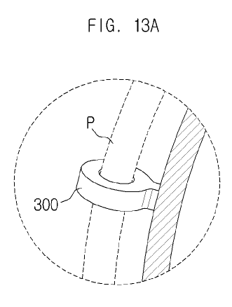

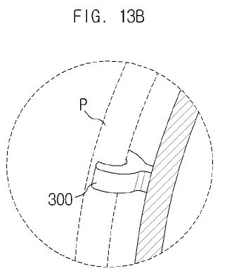

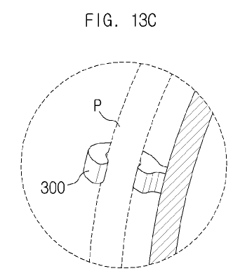

FIG. 12 illustrates a cross-sectional view of a fastening unit according to one example embodiment of the present invention, and FIGS. 13A through 13C illustrate magnifying perspective views of a fastening unit according to various example embodiments of the present invention. Referring to FIGS. 12 through 13C, the upper part 200, the inserting hole 210, the lower part 220 and a fastening unit 300 are illustrated.

As described above, the injection tube and/or the exhaust tube through which air travels between a human body and an external space can be formed integrally with the trocar or simply attached inside the trocar.

Also, as illustrated in FIG. 12, the tube may not be installed within the trocar but only the fastening unit 300 for fixing the tube can be formed within the trocar. The tube, P in FIGS. 12 through 13C, such as the injecting tube and/or the exhaust tube can be used after the tube is fastened to the fastening unit 300.

The fastening unit 300 is used to fasten a tube and can be formed in a variety of shape and structure such that a tubular member such as a ring (refer to FIG. 13A), a groove (refer to FIG. 13B) and a hook (see FIG. 13C) can be fastened.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

What is claimed is:

1. A trocar for a single port surgery installed on a skin puncture site of a patient to provide a pathway for insertion of a plurality of surgical instruments, the trocar comprising:
an upper part;
a plurality of inserting holes configured to be positioned on the upper part, the plurality of the surgical instruments being inserted thereto; and
a lower part configured to be formed in a tubular shape to connect to the upper part, the lower part being formed in an elastic material so that the lower part is bent when being inserted into the skin puncture site and is straightened after being introduced into the skin puncture site, wherein an open interior space is provided in which the inserting holes are connected with one another so that changing locations of the plurality of the surgical instruments is possible after the plurality of the surgical instruments are respectively inserted into the plurality of the inserting holes.

2. The trocar for the single port surgery according to claim 1, further comprising a valve configured to engage with the inserting hole to prevent an air flow.

3. The trocar for the single port surgery according to claim 1, wherein the upper part includes a cover configured to cover the inserting hole, the cover having a hole formed therein.

4. The trocar for the single port surgery according to claim 1, wherein the upper part is formed in a flexible material and the upper part further includes an upper part ring for maintaining a shape of the upper part.

5. The trocar for the single port surgery according to claim 1, wherein the lower part is formed in a flexible material and the lower part further includes a lower part ring for maintaining a shape of the lower part.

6. The trocar for the single port surgery according to claim 1, further comprising an injection tube configured to inject a gas from an external environment into inside of the skin puncture site.

7. The trocar for the single port surgery according to claim 6, further comprising an exhaust tube configured to exhaust a gas from the inside of the skin puncture site to the external environment.

8. The trocar for the single port surgery according to claim 7, further comprising an opening/closing valve configured to engage with at least one of the injection tube and the exhaust tube to control a flow of the gas.

9. The trocar for the single port surgery according to claim 8, further comprising:
a sensor configured to engage with a first end of the lower part and configured to detect the gas in the inside of the skin puncture site to produce a valve opening/closing signal; and
a valve opening/closing device configured to determine whether to operate the opening/closing valve based on the valve opening/closing signal received from the sensor.

10. The trocar for the single port surgery according to claim 7, wherein at least one of the injection tube and the exhaust tube extends to a first end of the lower part.

11. The trocar for the single port surgery according to claim 1, wherein the upper part and the lower part are separable from each other.

12. The trocar for the single port surgery according to claim 11, further comprising an engaging ring configured to engage a first end of the upper part with a second end of the lower part.

13. The trocar for the single port surgery according to claim 12, wherein the engaging ring includes,
an internal ring; and
an external ring configured to engage with the internal ring along an outer periphery of the internal ring.

14. The trocar for the single port surgery according to claim 13, wherein the internal ring and the external ring are engaged with each other in a concave-convex engagement.

15. The trocar for the single port surgery according to claim 12, wherein the engaging ring includes,
an upper part ring configured to engage with the upper part; and
a lower part ring configured to engage with the lower part, the lower part ring being drawn to the upper part ring by an attractive force caused by magnetism.

16. The trocar for the single port surgery according to claim 1, further comprising a fixing ring configured to enclose a surrounding of the lower part to fix a state of the lower part being inserted through the skin puncture site.

17. The trocar for the single port surgery according to claim 1, wherein the inserting hole has a certain level of a hardness such that the inserting hole is not distorted or deformed by an external force during a use of the trocar.

18. The trocar for the single port surgery according to claim 17, wherein the inserting hole is engaged with a distortion preventing member having the certain level of the hardness.

19. The trocar for the single port surgery according to claim 1, further comprising a valve configured to engage with the inserting hole to block an air flow, wherein a portion of the valve to be engaged with the inserting hole is configured to have a certain level of a hardness such that the inserting hole is not distorted or deformed by an external force during a use of the trocar.

20. The trocar for the single port surgery according to claim 1, wherein the upper part and the lower part are integrally formed with each other, and wherein a fastening member is formed inside the trocar to fasten a tube that provides a passageway for an air between an external environment and inside of the skin puncture site.
